SEQUENCE LISTING (22 sequences)

SEQ ID NO: 1 - DNA, 2800 bp, Homo sapiens
  source 1..2800: mol_type = genomic DNA
taatacgactcactataaggagaaagcttacatttgcttctgacacaactgtgttcactagcaacctcaaacagacaccatggagacagacacactcctgctatgggtactgctgctctgggttccaggttccactggcgccgctagcgatattcagatgacacagtcaccgagctccttgtctgcaagcgtgggggacagggttaccattacttgccgggcatctcaggacgttaacaccgcagttgcatggtaccagcagaagcccggtaaagcaccgaaactcttgatctactcagcaagtttcttggagtctggcgtaccaagtagattcagcggttccagatcaggtactgatttcacgcttacaatttctagcttgcaacccgaggatttcgcgacttactactgccagcaacactatacaacaccccctacttttgggcaggggactaaagtcgagataaaaggcggcggtggatctggtggaggtggaagcggcggaggtggctcagaagtacaacttgttgagtccggtggtggactggtccaacctggcggttcacttaggctgagttgcgctgcatcaggttttaatatcaaggacacttacatacattgggtccggcaggctccaggaaaaggactggaatgggtcgcccggatttatccaaccaatggatatacaaggtatgcggattcagttaaaggaagatttactatttccgccgatacgtccaaaaataccgcttacctccagatgaatagtcttagagctgaggacaccgccgtctattattgctcaaggtggggtggagatgggttttacgctatggatgtatgggggcagggcaccctcgttaccgtttcaagcgggggaggcgggtctgggggaggcggaagtgggggaggaggaagcgaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttgtctagagaaaacctgtattttcagggcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 2 - protein, 763 aa, Homo sapiens
METDTLLLWVLLLWVPGSTGAASDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 60 bp, Homo sapiens
  source 1..60: mol_type = genomic DNA
atggagacagacacactcctgctatgggtactgctgctctgggttccaggttccactggc

SEQ ID NO: 4 - protein, 20 aa, Homo sapiens
METDTLLLWVLLLWVPGSTG

SEQ ID NO: 5 - DNA, 726 bp, Homo sapiens
  source 1..726: mol_type = genomic DNA
gatattcagatgacacagtcaccgagctccttgtctgcaagcgtgggggacagggttaccattacttgccgggcatctcaggacgttaacaccgcagttgcatggtaccagcagaagcccggtaaagcaccgaaactcttgatctactcagcaagtttcttggagtctggcgtaccaagtagattcagcggttccagatcaggtactgatttcacgcttacaatttctagcttgcaacccgaggatttcgcgacttactactgccagcaacactatacaacaccccctacttttgggcaggggactaaagtcgagataaaaggcggcggtggatctggtggaggtggaagcggcggaggtggctcagaagtacaacttgttgagtccggtggtggactggtccaacctggcggttcacttaggctgagttgcgctgcatcaggttttaatatcaaggacacttacatacattgggtccggcaggctccaggaaaaggactggaatgggtcgcccggatttatccaaccaatggatatacaaggtatgcggattcagttaaaggaagatttactatttccgccgatacgtccaaaaataccgcttacctccagatgaatagtcttagagctgaggacaccgccgtctattattgctcaaggtggggtggagatgggttttacgctatggatgtatgggggcagggcaccctcgttaccgtttcaagc

SEQ ID NO: 6 - protein, 242 aa, Homo sapiens
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 7 - DNA, 321 bp, Homo sapiens
  source 1..321: mol_type = genomic DNA
gatattcagatgacacagtcaccgagctccttgtctgcaagcgtgggggacagggttaccattacttgccgggcatctcaggacgttaacaccgcagttgcatggtaccagcagaagcccggtaaagcaccgaaactcttgatctactcagcaagtttcttggagtctggcgtaccaagtagattcagcggttccagatcaggtactgatttcacgcttacaatttctagcttgcaacccgaggatttcgcgacttactactgccagcaacactatacaacaccccctacttttgggcaggggactaaagtcgagataaaa

SEQ ID NO: 8 - protein, 107 aa, Homo sapiens
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIK

SEQ ID NO: 9 - DNA, 45 bp, synthetic construct
  source 1..45: mol_type = other DNA
ggcggcggtggatctggtggaggtggaagcggcggaggtggctca

SEQ ID NO: 10 - protein, 15 aa, synthetic construct
GGGGSGGGGSGGGGS

SEQ ID NO: 11 - DNA, 359 bp, Homo sapiens
  source 1..359: mol_type = genomic DNA
gaagtacaacttgttgagtccggtggtggactggtccaacctggcggttcacttaggctgagttgcgctgcatcaggttttaatatcaaggacacttacatacattgggtccggcaggctccaggaaaaggactggaatgggtcgcccggatttatccaaccaatggatatacaaggtatgcggattcagttaaaggaagatttactatttccgccgatacgtccaaaaataccgcttacctccagatgaatagtcttagagctgaggacaccgccgtctattattgctcaaggtggggtggagatgggttttacgctatggatgtatgggggcagggcaccctcgttaccgtttcaag

SEQ ID NO: 12 - protein, 120 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 13 - DNA, 45 bp, synthetic construct
  source 1..45: mol_type = other DNA
gggggaggcgggtctgggggaggcggaagtgggggaggaggaagc

SEQ ID NO: 14 - DNA, 747 bp, Homo sapiens
  source 1..747: mol_type = genomic DNA
gaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagtgggggcggggggagtggtggcggaggaagtggcggggggggatctcaagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttg

SEQ ID NO: 15 - protein, 249 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 16 - DNA, 375 bp, Homo sapiens
  source 1..375: mol_type = genomic DNA
gaagttcagctgctcgaatccggcggcggccttgttcagccaggtggtagcttgaggctcagttgtgctgcatctgggtttacattctcaacttatgcgatgaactgggtgaggcaagcacctggaaagggacttgagtgggtctcaagaattcgctccaaatacaacaactatgcgacgtattacgcagactcagtgaaaggacggtttacgatatcacgggacgattcaaagaatacactgtatttgcagatgaattctcttagggccgaagacactgccgtatactattgtgtacgccacggtaattttggcaatagctatgtatcttggttcgcgtactggggccaaggcacccttgttactgtgtctagt

SEQ ID NO: 17 - protein, 125 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 18 - DNA, 45 bp, synthetic construct
  source 1..45: mol_type = other DNA
gggggcggggggagtggtggcggaggaagtggcggggggggatct

SEQ ID NO: 19 - DNA, 327 bp, Homo sapiens
  source 1..327: mol_type = genomic DNA
caagcggtggttactcaagagccctcccttactgtttctccgggcgggacggtcaccttgacttgtggcagttcaacaggggcagtcactactagtaattatgcgaattgggtccaagaaaagccgggccaagctttccggggactcatcggaggaacaaataaaagggcacccggcacacccgcgcgcttttccgggagtcttctgggcggcaaggcagccctcactctctctggggctcaacctgaggacgaggctgagtactattgtgccctctggtactcaaacctgtgggtctttggagggggaaccaagcttacggtcttg

SEQ ID NO: 20 - protein, 109 aa, Homo sapiens
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 21 - DNA, 705 bp, Homo sapiens
  source 1..705: mol_type = genomic DNA
tctagagaaaacctgtattttcagggcacccacacgtgccccccttgcccagcacccgaagccgcaggtggcccatcagtgtttctttttcctccaaaaccaaaagacacactcatgatctcccggacgcctgaggtgacctgtgtagtcgtagacgtatcccatgaggaccctgaagtaaagtttaactggtatgtagacggtgtggaagtacacaatgccaagactaaaccaagagaggaacagtataacagcacctatagggtagtttccgtgctcaccgttctccaccaagattggcttaacggtaaagaatataaatgtaaggtgtcaaataaggcactcggagccccgatcgaaaagaccatctctaaagcaaaaggacagcccagggagccacaagtctacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 22 - protein, 234 aa, Homo sapiens
SRENLYFQGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0193374 A1
(43) Pub. Date: Jul. 9, 2026

(54) HER-2-CD3-EPSILON BISPECIFIC ANTIBODIES

(71) Applicant: IntraAb, Inc., Richmond, CA (US)

(72) Inventors: Lijun Wu, Berkeley, CA (US); Vita Golubovskaya, Pinole, CA (US)

(21) Appl. No.: 19/435,104

(22) Filed: Dec. 29, 2025

Related U.S. Application Data
(63) Continuation of application No. PCT/US2024/035891, filed on Jun. 27, 2024.
(60) Provisional application No. 63/511,052, filed on Jun. 29, 2023.

(51) Int. Cl. C07K 16/32; A61K 48/00; A61P 35/00; C07K 16/28

(52) U.S. Cl. CPC C07K 16/32; A61K 48/0033; A61P 35/00; C07K 16/2809; C07K 2317/30

(57) ABSTRACT

The present invention is directed to bispecific Her-2-CD3 epsilon (CD3e) antigen-binding molecules. The present invention is further directed to a method for treating Her-2-positive cancer cells by administering the bispecific Her-2-CD3e antigen-binding molecule to the tumors. This invention provides a method of antibody production in cells and inside tumors with high in vivo efficacy through intratumoral delivery of mRNA encapsulated-lipid nanoparticles, wherein the mRNA encodes the antibody.

### CROSS-REFERENCE TO RELATED APPLICATION(S)

This application is a continuation of PCT/US2024/035891, filed Jun. 27, 2024; which claims priority to U.S. Provisional Application No. 63/511,052, filed Jun. 29, 2023. The contents of the above-identified applications are incorporated herein by reference in their entirety.

### REFERENCE TO SEQUENCE LISTING, TABLE OR COMPUTER PROGRAM

This application contains an ST.26 compliant Sequence Listing, which was submitted in xml format via Patent Center and is hereby incorporated by reference in its entirety. The .xml copy, created on Jun. 27, 2024, is named 119995-8056WO01SequenceListing.xml and is 27700 bytes in size.

### TECHNICAL FIELD

The present invention relates to Her-2-CD3 epsilon chain (CD3e) bispecific antibodies. The present invention also relates to a method for killing Her-2 positive cancer cells by transfecting mRNA-lipid nanoparticles (LNP) encoding the bispecific antibodies. Intratumoral delivery of Her-2-CD3 human Fc bispecific mRNA-LNP antibodies is effective to kill Her-2 positive cancer.

### BACKGROUND

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Using bispecific antibodies binding T cells and tumor associated antigen is the most common approach to design bispecific antibody by bringing cytotoxic T cells to kill cancer cells (1-3). Bispecific antibodies can be infused into patients by different routes such as intravenous, intraperitoneal and intratumorally. The advantage of bispecific antibodies compared with chemotherapy or monoclonal antibody is that it specifically targets antigen-positive cancer cells and simultaneously activates T cells (4).

HER2 is an 185 kDa transmembrane tyrosine kinase protein that belongs to the Erb B family of receptor tyrosine kinases (5). Four members of the human epidermal growth factor receptor (HER) family (EGFR family have been identified: EGFR (ERBB1, HER1), HER2 (ERBB2), HER3 (ERBB3), and HER4 (ERBB4). Their specific activating ligands are collectively known as neuregulins (NRG). In some solid tumors, the HER2/neu gene is amplified leading to an increased expression of the HER2 protein and increasing proliferation of tumor cells, driving drug resistance and increasing invasion and metastasis. Breast, gastric, bladder, and non-small cell lung cancers can have Her-2 amplification that is associated with poor prognosis. Tumor cells with high levels of HER2 have a more aggressive phenotype.

Breast cancer is the most frequently occurring malignancy in women with about 212,600 cases diagnosed per year in the United States and Her-2 is overexpressed in 20-30% of breast cancer patients (6). The first-line therapy approved for HER2 positive tumors is Trastuzumab (Herceptin) and Pertuzumab linked to taxane and further treatment with an antibody-drug conjugate to achieve satisfactory outcomes (6). Trastuzumab (FDA approved in 1998) consists of the antigen-binding fragment (Fab) of the humanized murine 4D5 mAb, directed against the extracellular domain (ECD) of HER-2, spliced to the Fc fragment of human IgG. Lapatinib (Her-2 tyrosine kinase inhibitor) was approved by the FDA in 2007 for HER2 positive breast cancer (6). It is important to develop novel anti-Her-2 agents to overcome acquired drug-resistance of Her-2-positive tumors.

Her-2 sequence can be found in Uniport database P04626-1. Her-2 is a polypeptide of 1255 amino acids, consisting of extracellular domain of 23-652 amino acids, transmembrane domain of 653-675 amino acids; and cytoplasmic domain of 676-1255 amino acids. Extracellular domain contains 4 subdomains. Her-2 does not have a ligand but it heterodimerizes with EGFR and other members of human epidermal growth factor receptor (HER) family to bind ligands and drive survival signaling in tumors.

### BRIEF DESCRIPTION OF THE DRAWINGS

FIG. 1 shows the structure of Her-2 ScFv-linker-CD3 ScFv-human Fc mutant. The antibody has one Her-2 binding moiety, one CD3 binding moiety, and a human Fc structure optionally with L234A, L235A and P329G mutations in CH2 region of Fc. One DNA construct is used, which will form duplex due to connection in human Fc hinge region.

FIG. 2 shows the scheme of DNA vector template (top) used for in vitro transcription of mRNA (bottom). 5′UTR, 5′ untranslated region; 3′UTR, 3′untranslated region; poly A tail: for increased stability.

FIG. 3 shows the transfection of Her-2-CD3 hFc mRNA-LNP into cancer cell lines generated functional antibody that bound to Her-2-positive cells and CD3-positive T cells.

FIG. 4 shows that Her-2-CD3 mRNA-LNP transfected A1847 ovarian cancer cells generated bispecific antibody that killed Her-2 positive cancer cells in a dose-dependent manner. RTCA killing assay was performed with supernatant at different dilutions from A1847 Her-2-CD3 hFc mRNA-LNP-transfected cancer cells.

FIG. 5 shows that supernatant from A1847 Her-2 mRNA transfected cells with T cells secreted IFN-gamma against Her-2-positive target cells.

FIG. 6 shows that Her-2-CD3 hFc mRNA-LNP significantly decreased OVCAR-5 tumor growth.

### DETAILED DESCRIPTION

### Definitions

As used herein, “bispecific antibody” is an artificial protein that can simultaneously bind to two different types of antigen or different epitopes of the same antigen.

As used herein, “CD3 epsilon (CD3e)” is a polypeptide encoded by the CD3E gene which resides on chromosome 11. CD3-epsilon polypeptide, which together with CD3-gamma, -delta and -zeta, and the T-cell receptor alpha/beta forms the T cell receptor-CD3 complex. This complex plays an important role in coupling antigen recognition to several intracellular signal-transduction pathways. The CD3 epsilon polypeptide plays an essential role in T-cell development. CD3 epsilon, CD3e, and CD3 are used interchangeably in this application.

As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for preparing a scFv are known to a person skilled in the art.

As used herein, a “tumor antigen” means a biological molecule having antigenicity, expression of which causes cancer.

### Bispecific Antigen-Binding Molecule

The present invention is directed to bispecific antibodies or bispecific antigen-binding molecules that specifically bind to both human Her-2 and human CD3e. The bispecific antibody or bispecific antigen-binding molecule targets Her-2 tumor antigen which is highly overexpressed in many types of cancer such as ovarian, pancreatic, and colon cancer. The Her-2-CD3 bispecific antibody or antigen-binding molecule of the present invention have high cytotoxic activity against Her-2-positive ovarian cancer cell line and does not have activity with Her-2-negative cell line. The bispecific antibody activates T cells and re-directs T cells to Her-2-positive cancer cells to kill cancer cells.

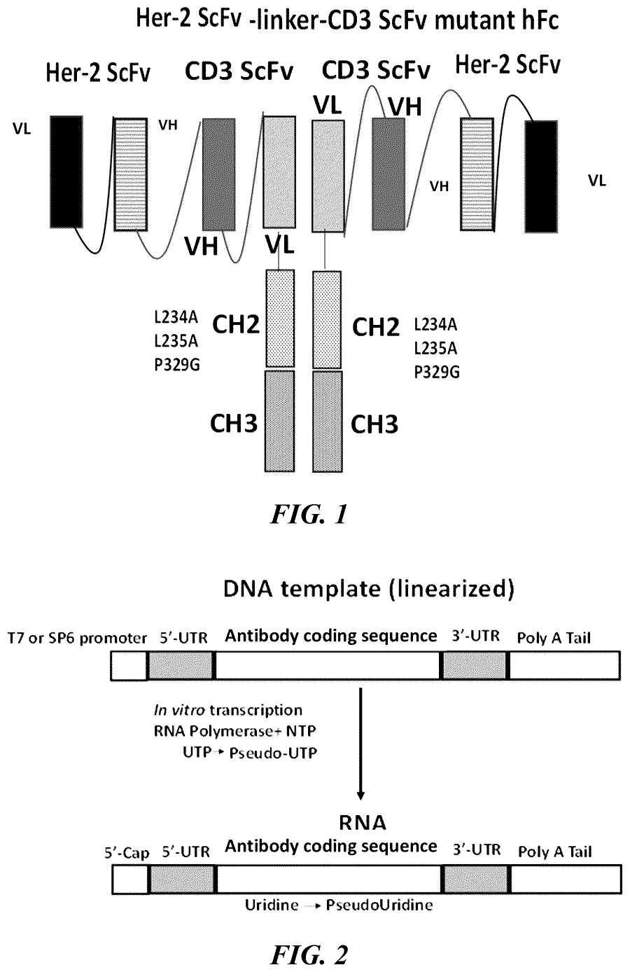

FIG. 1 illustrates one embodiment of the bispecific antibody structure of the present invention (HER-2 ScFv-CD3 ScFv-human Fc). FIG. 1 shows a monovalent humanized HER-2 ScFv linked to a monovalent CD3e ScFv, which is linked to human Fc; the structure consists of one DNA construct. The structure has an HER-2 scFv and CD3e scFv, which are connected by a linker and then CD3e Scfv is fused to human Fc for increased stability. The bispecific antibody (BITE-human Fc format) comprises one binding moiety to HER-2, and one binding moiety to CD3 epsilon. The antibody has dimeric conformation. In one embodiment, the human Fc has L234A, L235A, P329G mutations to inhibit Fc-dependent activity activating NK cells, and to decrease toxicity due to Fc-dependent antibody-dependent cellular cytotoxicity (ADCC) immune response. The amino acid numbers (L234, L235, and P329) in CH2 of the Fc-region are counted from human IgG1 according to [1].

The present bispecific antigen-binding molecules target Her-2 tumor antigen, which is highly overexpressed in many types of cancer such as ovarian, lung, breast, and colon cancer. The Her-2-CD3 bispecific antibodies of the present invention have high cytotoxic activity against Her-2-positive ovarian cancer cell line and do not have activity with Her-2-negative ell line. The bispecific antibody activates T cells and re-directs T cells to Her-2-positive cancer cells to kill cancer cells.

In one embodiment, the Fc domain comprises one or more amino acid substitution that reduces binding to an Fc receptor and/or reduces effector function. In one embodiment, the one or more amino acid substitutions in the Fc domain are selected from the group consisting of L234, L235, and P329 (Kabat numbering). In one embodiment, the said amino acid substitutions are L234A, L235A and P329G.

In one embodiment, Fc mutations L234A, L235A, and P329G mutations are used to prevent Fc-dependent immune reactions.

In one embodiment, Her-2 ScFv is a high affinity derivative from Herceptin 4D5 antibody.

In one embodiment, the VL of the Her-2 antibody has the amino acid sequence of SEQ ID NO: 8, and the VH of the Her-2 antibody has the amino acid sequence of SEQ ID NO: 12.

The present invention is directed to a bispecific antigen-binding molecule comprising Her-2 VL having SEQ ID NO: 8, a first linker, Her-2 VH having SEQ ID NO: 12, a second linker, CD3 VH having SEQ ID NO: 17, a third linker, CD3 VL having SEQ ID NO: 20, and a human Fc domain.

In one embodiment, the human Fc domain comprises a hinge linker, and CH2-CH3 of human IgG1, optionally substituted with one or more amino acids in Fc.

In one embodiment, the linkers of the present bispecific antigen-binding molecules have the amino acid sequence of (GGGGS)n, and n=1-5. In one preferred embodiment, the linker has the amino acid sequence of (GGGGS)₃.

In one embodiment, the bispecific antigen-binding molecule does not contain CH2 or CH3 for shorter sequence and shorter half-life. Such bispecific antigen-binding molecule comprises Her-2 VL having SEQ ID NO: 8, a first linker, Her-2 VH having SEQ ID NO: 12, a second linker, CD3 VH having SEQ ID NO: 17, a third linker, CD3 VL having SEQ ID NO: 20

The present invention provides an isolated DNA sequence comprising: (a) a promoter coding sequence, (b) 5′-UTR (untranslated region) coding sequence, (c) a coding sequence to encode the bispecific antigen-binding molecule of the present invention, (d) a 3′-UTR coding sequence, and (e) a poly A tail sequence.

In the DNA sequence, the promoter is T7, T7AG promoter, or SP6 promoter. Poly A tail sequence is from 20-170 nucleotides. Poly A tail sequence optionally comprises one or more linkers in between the poly A segments. If poly A tail is longer than 60 nucleotides, then it typically contains a linker which includes non-adenosine nucleotides. A linker is 5-30 or 5-25 nucleotides, e.g., 10 nucleotides or 20 nucleotides. In one example, poly A tails is 110 nucleotides in length, consisting of a stretch of 30 adenosine residues, followed by a 10-nucleotide linker sequence, and another 70 adenosine residues. In another example, poly A tails is 90 nucleotides in length, consisting of a stretch of 40 adenosine residues, followed by a 30-nucleotide linker sequence, and another 30 adenosine residues. In yet another example, poly A tail is 150-160 nucleotides in length, consisting of a two linker sequences.

DNA expression is finely regulated at the post-transcriptional level. Untranslated regions are not translated into amino acids; however, UTRs of mRNAs may control their translation, degradation and localization include stem-loop structures, upstream initiation codons and open reading frames, internal ribosome entry sites and various cis-acting elements that are bound by RNA-binding proteins. UTRs are important in the post-transcriptional regulation of DNA expression, including modulation of the transport of mRNAs out of the nucleus and of translation efficiency, subcellular localization, and stability.

5′-UTR typically has 10-1000 nucleotides, or 20-500 nucleotides, or 30-200 nucleotides, or 30-100 nucleotides. For example, 5′-UTR is 50 nucleotides. 3′-UTR typically has 10-3000 nucleotides, for example, 50-500 nucleotides, or 100-300 nucleotides. Preferred 5′-UTRs and 3′-UTRs are UTRs of β-globin, or UTRs of Pfizer COVID vaccine.

β-Globin gene is shown in:
www.ncbi.nlm.nih.gov/nucleotide/V00497.1?report=genbank&log$=nuclalign&blast_rank=5&RID=TDDZ1K98016

In one embodiment, the 5′-untranslated region is derived from human alpha-globin RNA with an optimized Kozak sequence. The 3′ untranslated region comprises two sequence elements derived from the amino-terminal enhancer of split (AES) mRNA and the mitochondrial encoded 12S ribosomal RNA to confer RNA stability and high total protein expression.

Any suitable vector, such as Vector pSP64 Poly(A) (Promega) or pGEM3Z-Vector (Promega) can be used as a cloning vector for the DNA sequence described above.

For example, to engineer the pEM3Z-β-globin UTR-UTR-poly A tail, the 3′-UTR of the β-globin molecule flanked by restriction enzyme site can be amplified from human bone marrow. For example, a single (pEM3Z-1β-globin-UTR-A [120]) or 2 serial fragments (pEM3Z-2β-globin-UTR-A[120]) can be inserted in front of the poly(A) tail.

The present invention provides a method for producing the bispecific antigen-binding molecule in cells and, also in vivo inside the body. The method comprises the steps of: obtaining the DNA sequence as described above, transcribing the DNA sequence to mRNA with RNA polymerase in vitro, encapsulated RNA into lipid nanoparticles (LNPs), and transfecting the mRNA into cells or into mice xenograft tumors, and translating the mRNA in the cells to produce the bispecific antigen-binding fragment thereof.

FIG. 2 shows the scheme of DNA vector template (top) used for in vitro transcription of RNA (bottom). The mRNA comprises (i) 5′-UTR (untranslated region) coding sequence, (ii) the antibody coding sequence, (iii) a 3′-UTR coding sequence, and (iv) a poly A tail sequence.

mRNA can be embedded to LNP to transfect human cells.

In one embodiment, LNPs comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-distearoyl-sn-glycero-3-phosphoethanolamine-N-[maleimide(polyethylene glycol)-2000](DSPE-PEG2000-MAL). [LNP-102-MAL (i)]

In one embodiment, LNPs comprise 8-[(2-hydroxyethyl)[6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000). [LNP-102 (ii)]

In one embodiment, LNPs comprise 2-hexyl-decanoic acid, 1,1′-[[(4-hydroxybutyl)imino]di-6,1-hexanediyl]ester (ALC-0315), DSPC, Cholesterol, and α-[2-(ditetradecylamino)-2-oxoethyl]-ω-methoxy-poly(oxy-1,2-ethanediyl) (ALC-0159). [LNP-315]

Insertion of mRNA into LNPs provides protection of mRNA from degradation and increases the stability mRNA; mRNA is then released from LNPs into cells in vivo to generate protein. mRNA-lipid nanoparticle preparation is described in Schoenmaker (International J. Pharmaceutics, 601: 120856, 2021), the article is incorporated herein by reference in its entirety, in particular regarding the LNPs.

In a preferred embodiment, the mRNA further comprises 5′-cap, which is 5′-end to 5′-UTR. 5′-cap stabilizes mRNA. In addition to its essential role of cap-dependent initiation of protein synthesis, the mRNA cap also functions as a protective group from 5′ to 3′ exonuclease cleavage and a unique identifier for recruiting protein factors for pre-mRNA splicing, polyadenylation and nuclear export. It also acts as the anchor for the recruitment of initiation factors that initiate protein synthesis and the 5′ to 3′ looping of mRNA during translation.

The mRNA is transcribed with RNA polymerase in vitro from a DNA sequence comprising (a) a promoter coding sequence, (b) the 5′-UTR coding sequence, (c) the CAR coding sequence, (d) the 3′-UTR coding sequence, and (e) the poly A tail sequence. The poly A tail sequence improves stability and protein translation.

### Method for Treating Cancer

The present invention is directed to a method for treating cancer, comprising the step of administering a bispecific Her-2-CD3 antibody binding molecule to a subject suffering from cancer, wherein the cancer is selected from the group consisting of ovarian cancer, lung cancer, pancreatic cancer, stomach cancer, and cervical cancer, and the cancer is Her-2-positive.

The present method redirects the activity of T cells, independently of their T cell receptor (TCR) specificity, by bispecific Her-2-CD3 antibody, to treat cancer. The method is based on recognition of a tumor cell surface antigen and simultaneous binding to the CD3 epsilon chain (CD3e) within the T-cell receptor (TCR) complex on T cells. This triggers T-cell activation, including release of cytotoxic molecules, cytokines and chemokines, and induction of T-cell proliferation.

In the present method, mRNA-LNP is used to produce the bispecific antibodies inside tumors in vivo, which allows to produce and manufacture antibodies inside organism in vivo and kill tumors by bringing T cells to the tumor site. The present method produces bispecific antibodies intratumorally using mRNA-LNP and brings T cells to kill tumors.

Different immunomodulators can be added to increase activity of T cells and other immune cells for higher efficacy of the therapy.

This application demonstrates that Her-2-CD3e bispecific antigen-binding molecule can be produced using in vitro transcription of DNA template to produce antibody using RNA-LNP transfected into 293 cells.

Antibodies also can be produced using adenoviruses or other viruses inside the cells providing in vivo manufacturing which decreases cost of manufacturing, generating stable cell lines for production of antibodies.

This application demonstrates the efficacy of the bispecific antigen-binding molecule targeting Her-2 antigen which is overexpressed in cancer tumors. This application demonstrates that Her-2-CD3e bispecific antigen-binding molecule binds CD3e antigen and Her-2 antigen. This bispecific antigen-binding molecule delivered with T cells specifically decreases viability of Her-2-positive ovarian cancer cells, but not Her-2-negative ovarian cancer cells. Her-2-CD3e mutant Fc antibody delivered with T cells causes secretion of significant level of IFN-gamma after co-incubation with Her-2-positive ovarian cancer cells but not after co-incubation with Her-2-negative target cells. The application demonstrates that Her-2-CD3-mutant hFc antibody RNA-LNPs injected into A1847 tumors (intratumorally) with T cells significantly decreases xenograft tumor growth. This implies in vivo production of antibody from injected RNA-LNP and shows its high in vivo efficacy.

The application demonstrates that Her-2 CD3 bispecific antigen-binding molecules with T cells significantly kill Her-2-positive cancer cells, but do not kill Her-2-negative cells. This implies high specificity of the bispecific antigen-binding molecule.

The following examples further illustrate the present invention. These examples are intended merely to be illustrative of the present invention and are not to be construed as limiting.

### EXAMPLES

### Example 1. Materials and Methods

### Cells and Culture Medium

HEK293 S cells from AlStem (Richmond, CA) were cultured in Dulbecco's Modified Eagle's Medium (DMEM) plus 10% FBS and 1% penicillin/streptomycin. Human peripheral blood mononuclear cells (PBMC) were isolated from whole blood obtained from the Stanford Hospital Blood Center, Stanford, CA according to IRB-approved protocol using Ficoll-Paque solution (GE Healthcare). T cells were expanded from PBMC with CD3-CD28 beads in medium with IL-2 as described below (7). Ovarian cancer cell lines: Her-2-positive: A1847 cells and SKOV-3 obtained from ATCC were used for the study. The cells were cultured in a humidified 5% CO₂ (8).

### Antibodies

The (APC)-labeled anti-CD3 and secondary antibodies were described in (9).

### Preparation of LNP-102 and LNP-315

SM-102: 8-[(2-hydroxyethyl)[6-oxo-6-(undecyloxy)hexyl]amino]-octanoic acid, 1-octylnonyl ester; CAS number: 2089251-47-6
ALC-0315: 2-hexyl-decanoic acid, 1,1′-[[(4-hydroxybutyl)imino]di-6,1-hexanediyl]ester
CAS number 2036272-55-4.
ALC-0159: α-[2-(ditetradecylamino)-2-oxoethyl]-ωhoxy-poly(oxy-1,2-ethanediyl)
CAS: 1849616-42-7
DMG-PEG2000: 1,2-dimyristoyl-rac-glycero-3-methoxypolyethylene glycol-2000
DSPC: Distearoylphosphatidylcholine

### LNP-102 Formulation:

LNP-102 is a lipid nanoparticle (LNP) formulated with (i) SM-102, DSPC, Cholesterol, and DSPE-PEG2000-MAL, or (ii) SM-102, DSPC, Cholesterol, and DMG-PEG2000.

LNP-102-MAL (i) is formulated at a molar ratio of SM-102, DSPC, Cholesterol, and DSPE-PEG2000-MAL 40:15:40:0.5 mol %, 40:15:40:0.75 mol %, 40:15:40:1.5 mol %, or 40:15:40:5 mol %. LNP was used at 10 mg/ml concentration.

LNP-102 (ii) is formulated at molar ratio of SM-102, DSPC, Cholesterol, and DMG-PEG2000 (40:15:40:1.5 mol %). LNP was used at 10 mg/ml concentration.

### LNP-315 Formulation:

LNP-0315 is a lipid nanoparticle (LNP) formulated with ALC-0315, DSPC, Cholesterol, and ALC-0159 at a ratio 46.3: 9.4: 42.7: 1.6 mol %, respectively. LNP-0315 is formulated at optimal molar ratio for efficient delivery of mRNA-based vaccines. LNP-0315 was used at 10 mg/mL stock solution in ethanol.

### RNA Encapsulation to LNP

12 ul of LNP mix (LNP102(ii)) was mixed with 36 ul RNA (10-25 ug) in 100 mM sodium acetate pH 4 and 48 ul CA and Mg-free PBS using microfluidics cartridge and Nanosystem. After mixing in Nanosystem, RNA encapsulated into LNP was used for transfection of 293 or PBS cells. The LNP size was 90-140 nm based on NanoSystem protocol.

In Vitro RNA Transcription of her-2-CD3 Mutant Human Fc Antibody and Expression of her-2-CD3 Mutant Human Fc Antibody.

RNA was transcribed with commercial in vitro transcription kit using DNA template with RNA polymerase promoter, cap1, 5′UTR, antibody sequence, 3′UTR, and poly A tail according to manufacturer's protocol.

Her-2-CD3-mutant hFc (PMC2087) was embedded to LNP-102 (ii), and Her-2-CD3-human Fc mRNA-LNPs were transfected to 293 or cancer cell lines. The supernatants were collected after 72-96 hours to test for binding and in vitro activity. For mouse xenograft study, Her-2-CD3-mutant hFc mRNA-LNP were injected intratumorally to mice and tested with injected human T cells for in vivo efficacy. LNP mix and transfection of RNA-LNP were prepared.

### PBMC

PBMC were resuspended at 1×10⁶ cells/ml in AIM V-AlbuMAX medium (Thermo Fisher) containing 10% FBS with 300 U/ml IL-2 (Thermo Fisher). PBMC cells were activated with CD3/CD28 Dynabeads (Invitrogen) and used for cytotoxicity analysis with bi-specific antibodies.

### Fluorescence-Activated Cell Sorting (FACS) Analysis

The allophycocyanin (APC)-labeled anti-CD3 (eBioscience, San Diego, CA) antibody was used for FACS analysis using FACSCalibur (BD Biosciences). For FACS with cancer cell lines to detect Her-2 levels bi-specific Her-2-CD3 antibody and Anti-human Fc antibodies were used and analyzed on FACSCalibur, as described (8).

### Real-Time Cytotoxicity Assay (RTCA)

Adherent ovarian cancer target cells (10,000 cells per well) were seeded into 96-well E-plates (Acea Biosciences, San Diego, CA) and cultured overnight using the impedance-based real-time cell analysis (RTCA) iCELLigence system (Acea Biosciences). After 20-24 hours, the medium was replaced with 1×10⁵ effector cells T cells, T cells with bispecific antibody or antibody alone in AIM V-AlbuMAX medium containing 10% FBS, in triplicate. The cells were monitored for >40 hours with the RTCA system, and impedance (proportional to cell index) was plotted over time. Cytotoxicity was calculated as (impedance of target cells without effector cells−impedance of target cells with effector cells)×100/impedance of target cells without effector cells.

### ELISA Assay for Cytokine Secretion

The target cells were cultured with the effector cells or agents at in U-bottom 96-well plates with AIM V-AlbuMAX medium plus 10% FBS, in triplicate. After 16 h the supernatant was removed and centrifuged to remove residual cells. In some experiments, supernatant after RTCA assay was used for ELISA cytokine assays. The supernatant was transferred to a new 96-well plate and analyzed by ELISA for human cytokines using kits from Thermo Fisher according to the manufacturer's protocol. The EC50 was calculated with GraphPad Prism software.

### Example 2. The Sequences of Humanized her-2-CD3e Human Fc Bispecific Antibody (FIG. 1)

FIG. 1 shows the structure of humanized Her-2 ScFv-linker-G4Sx3-CD3 ScFv mutant human Fc consisting of one DNA template construct for in vitro transcription.

### Pmc2087 Her-2-CD3 Mutant Fc

A DNA template for mRNA in vitro transcription includes T7 promoter (underlined bold below); 5′UTR (regular font), Her-2-CD3-human Fc Ab sequence in bold starts with ATG start codon (underlined) and ends with stop codon TGA (underlined); 3′UTR (regular font); >150 poly A tail with linker in the middle (italics).

(SEQ ID NO: 1)
TAATACGACTCACTATAAGgagaaagcttacatttgcttctgacacaact
gtgttcactagcaacctcaaacagacaccATGGAGACAGACACACTCCTG
CTATGGGTACTGCTGCTCTGGGTTCCAGGTTCCACTGGCGCCGCTAGCGA
TATTCAGATGACACAGTCACCGAGCTCCTTGTCTGCAAGCGTGGGGGACA
GGGTTACCATTACTTGCCGGGCATCTCAGGACGTTAACACCGCAGTTGCA
TGGTACCAGCAGAAGCCCGGTAAAGCACCGAAACTCTTGATCTACTCAGC
AAGTTTCTTGGAGTCTGGCGTACCAAGTAGATTCAGCGGTTCCAGATCAG
GTACTGATTTCACGCTTACAATTTCTAGCTTGCAACCCGAGGATTTCGCG
ACTTACTACTGCCAGCAACACTATACAACACCCCCTACTTTTGGGCAGGG
GACTAAAGTCGAGATAAAAGGCGGCGGTGGATCTGGTGGAGGTGGAAGCG
GCGGAGGTGGCTCAGAAGTACAACTTGTTGAGTCCGGTGGTGGACTGGTC
CAACCTGGCGGTTCACTTAGGCTGAGTTGCGCTGCATCAGGTTTTAATAT
CAAGGACACTTACATACATTGGGTCCGGCAGGCTCCAGGAAAAGGACTGG
AATGGGTCGCCCGGATTTATCCAACCAATGGATATACAAGGTATGCGGAT
TCAGTTAAAGGAAGATTTACTATTTCCGCCGATACGTCCAAAAATACCGC
TTACCTCCAGATGAATAGTCTTAGAGCTGAGGACACCGCCGTCTATTATT
GCTCAAGGTGGGGTGGAGATGGGTTTTACGCTATGGATGTATGGGGGCAG
GGCACCCTCGTTACCGTTTCAAGCGGGGGAGGCGGGTCTGGGGGAGGCGG
AAGTGGGGGAGGAGGAAGCGAAGTTCAGCTGCTCGAATCCGGCGGCGGCC
TTGTTCAGCCAGGTGGTAGCTTGAGGCTCAGTTGTGCTGCATCTGGGTTT
ACATTCTCAACTTATGCGATGAACTGGGTGAGGCAAGCACCTGGAAAGGG
ACTTGAGTGGGTCTCAAGAATTCGCTCCAAATACAACAACTATGCGACGT
ATTACGCAGACTCAGTGAAAGGACGGTTTACGATATCACGGGACGATTCA
AAGAATACACTGTATTTGCAGATGAATTCTCTTAGGGCCGAAGACACTGC
CGTATACTATTGTGTACGCCACGGTAATTTTGGCAATAGCTATGTATCTT
GGTTCGCGTACTGGGGCCAAGGCACCCTTGTTACTGTGTCTAGTGGGGGC
GGGGGGAGTGGTGGCGGAGGAAGTGGCGGGGGGGGATCTCAAGCGGTGGT
TACTCAAGAGCCCTCCCTTACTGTTTCTCCGGGCGGGACGGTCACCTTGA
CTTGTGGCAGTTCAACAGGGGCAGTCACTACTAGTAATTATGCGAATTGG
GTCCAAGAAAAGCCGGGCCAAGCTTTCCGGGGACTCATCGGAGGAACAAA
TAAAAGGGCACCCGGCACACCCGCGCGCTTTTCCGGGAGTCTTCTGGGCG
GCAAGGCAGCCCTCACTCTCTCTGGGGCTCAACCTGAGGACGAGGCTGAG
TACTATTGTGCCCTCTGGTACTCAAACCTGTGGGTCTTTGGAGGGGGAAC
CAAGCTTACGGTCTTGTCTAGAGAAAACCTGTATTTTCAGGGCACCCACA
CGTGCCCCCCTTGCCCAGCACCCGAAGCCGCAGGTGGCCCATCAGTGTTT
CTTTTTCCTCCAAAACCAAAAGACACACTCATGATCTCCCGGACGCCTGA
GGTGACCTGTGTAGTCGTAGACGTATCCCATGAGGACCCTGAAGTAAAGT
TTAACTGGTATGTAGACGGTGTGGAAGTACACAATGCCAAGACTAAACCA
AGAGAGGAACAGTATAACAGCACCTATAGGGTAGTTTCCGTGCTCACCGT
TCTCCACCAAGATTGGCTTAACGGTAAAGAATATAAATGTAAGGTGTCAA
ATAAGGCACTCGGAGCCCCGATCGAAAAGACCATCTCTAAAGCAAAAGGA
CAGCCCAGGGAGCCACAAGTCTACACCCTGCCCCCATCCCGGGATGAGCT
GACCAAGAACCAGGTCAGCCTGACCTGCCTGGTCAAAGGCTTCTATCCCA
GCGACATCGCCGTGGAGTGGGAGAGCAATGGGCAGCCGGAGAACAACTAC
AAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCAT
GCTCCGTGATGCATGAGGCTCTGCACAACCACTACACGCAGAAGAGCCTC
TCCCTGTCTCCCGGGAAATGAgctcgctttcttgctgtccaatttctatt
aaaggttcctttgttccctaagtccaactactaaactgggggatattatg
aagggccttgagcatctggattctgcctaataaaaaacatttattttcat
tgcagctcgctttcttgctgtccaatttctattaaaggttcctttgttcc
ctaagtccaactactaaactgggggatattatgaagggccttgagcatct
ggattctgcctaataaaaaacatttattttcattgcaGTCGACTCTAGAA
AAAAAAAAAAAAAAAAAAAAAAAAAAAAAAAAAAAAAAAGGATCCCCGGG
CGAGCTCCCAAAAAAAAAAAAAAAAAAAAAAAAAAAAAACCGAATTCCTG
CAGCTCGAGAAAAAAAAAAAAAAAAAAAAAAAAAAAAAAAAAAAAAAAAA

Amino Acid Sequence, her-2-CD3-Mutant Human Fc

Signal peptide underlined, Her-2 Scfv in bold, CD3 Scfv underlined in italics, mutant Fc with mutations in enlarged font and bold

(SEQ ID NO: 2)
METDTLLLWVLLLWVPGSTGAASDIQMTQSPSSLSASVGDRVTITC
RASQDVNTAVAWYQQKPGKAPKLLIYSASFLESGVPSRFSGSRSGT
DFTLTISSLQPEDFATYYCQQHYTTPPTFGQGTKVEIKGGGGSGGG
GSGGGGSEVQLVESGGGLVQPGGSLRLSCAASGFNIKDTYIHWVRQ
APGKGLEWVARIYPTNGYTRYADSVKGRFTISADTSKNTAYLQMNS
LRAEDTAVYYCSRWGGDGFYAMDVWGQGTLVTVSSGGGGSGGGGSG
GGGSEVQLLESGGGLVQPGGSLRLSCAASGFTFSTYAMNWVRQAPG
KGLEWVSRIRSKYNNYATYYADSVKGRFTISRDDSKNTLYLQMNSL
RAEDTAVYYCVRHGNFGNSYVSWFAYWGQGTLVTVSSGGGGSGGGG
SGGGGSQAVVTQEPSLTVSPGGTVTLTCGSSTGAVTTSNYANWVQE
KPGQAFRGLIGGTNKRAPGTPARFSGSLLGGKAALTLSGAQPEDEA
EYYCALWYSNLWVFGGGTKLTVLSRENLYFQGTHTCPPCPAPE [symbol] G
GPSVFLFPPKPKDTLMISRTPEVTCVVVDVSHEDPEVKFNWYVDGV
EVHNAKTKPREEQYNSTYRVVSVLTVLHQDWLNGKEYKCKVSNKAL [symbol]
APIEKTISKAKGQPREPQVYTLPPSRDELTKNQVSLTCLVKGFYP
SDIAVEWESNGQPENNYKTTPPVLDSDGSFFLYSKLTVDKSRWQQG
NVFSCSVMHEALHNHYTQKSLSLSPGK

### Signaling Peptide Nucleotide, SEQ ID NO: 3

ATGGAGACAGACACACTCCTGCTATGGGTACTGCTGCTCTGGGT
TCCAGGTTCCACTGGC

### Signaling Peptide, Amino Acid, SEQ ID NO: 4

METDTLLLWVLLLWVPGSTG

Nucleotide: GCCGCTAGC
Amino Acid: AAS

### Her-2 ScFv (VL-Linker-VH) Nucleotide, SEQ ID NO: 5

gatattcagatgacacagtcaccgagctccttgtctgcaagcgtggggga
cagggttaccattacttgccgggcatctcaggacgttaacaccgcagttg
catggtaccagcagaagcccggtaaagcaccgaaactcttgatctactca
gcaagtttcttggagtctggcgtaccaagtagattcagcggttccagatc
aggtactgatttcacgcttacaatttctagcttgcaacccgaggatttcg
cgacttactactgccagcaacactatacaacaccccctacttttgggcag
gggactaaagtcgagataaaaggcggcggtggatctggtggaggtggaag
cggcggaggtggctcagaagtacaacttgttgagtccggtggtggactgg
tccaacctggcggttcacttaggctgagttgcgctgcatcaggttttaat
atcaaggacacttacatacattgggtccggcaggctccaggaaaaggact
ggaatgggtcgcccggatttatccaaccaatggatatacaaggtatgcgg
attcagttaaaggaagatttactatttccgccgatacgtccaaaaatacc
gcttacctccagatgaatagtcttagagctgaggacaccgccgtctatta
ttgctcaaggtggggggagatgggttttacgctatggatgtatgggggca
gggcaccctcgttaccgtttcaagc

Her-2 scFv Amino Acid, SEQ ID NO: 6

DIQMTQSPSSLSASVGDRVTITCRASQDVNTAVAWYQQKPGKAPKLLIYS
ASFLESGVPSRFSGSRSGTDFTLTISSLQPEDFATYYCQQHYTTPPTFGQ
GTKVEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDVWGQGTLVTVSS

### Her-2 VL Nucleotide, SEQ ID NO: 7

Gatattcagatgacacagtcaccgagctccttgtctgcaagcgtggggga
cagggttaccattacttgccgggcatctcaggacgttaacaccgcagttg
catggtaccagcagaagcccggtaaagcaccgaaactcttgatctactca
gcaagtttcttggagtctggcgtaccaagtagattcagcggttccagatc
aggtactgatttcacgcttacaatttctagcttgcaacccgaggatttcg
cgacttactactgccagcaacactatacaacaccccctacttttgggcag
gggactaaagtcgagataaaa

### Her-2 VL Amino Acid, SEQ ID NO: 8

DIQMTQSPSSLSASVGDRVTITCRASQDVNTAVAWYQQKPGKAPKLLIYS
ASFLESGVPSRFSGSRSGTDFTLTISSLQPEDFATYYCQQHYTTPPTFGQ
GTKVEIK

### G4Sx3 Linker Nucleotide, SEQ ID NO: 9

GGCGGCGGTGGATCTGGTGGAGGTGGAAGCGGCGGAGGTGGCTCA

### G4Sx3 Linker Amino Acid, SEQ ID NO: 10

GGGGSGGGGSGGGGS

### Her-2 VH Nucleotide, SEQ ID NO: 11

Gaagtacaacttgttgagtccggtggtggactggtccaacctggc
ggttcacttaggctgagttgcgctgcatcaggttttaatatcaag
gacacttacatacattgggtccggcaggctccaggaaaaggactg
gaatgggtcgcccggatttatccaaccaatggatatacaaggtat
gcggattcagttaaaggaagatttactatttccgccgatacgtcc
aaaaataccgcttacctccagatgaatagtcttagagctgaggac
accgccgtctattattgctcaaggtggggggagatgggttttacg
ctatggatgtatgggggcagggcaccctcgttaccgtttcaagc

### Her-2 VH Amino Acid, SEQ ID NO: 12

EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDVWGQGTLVTVSS

### G4Sx3 Linker Nucleotide, SEQ ID NO: 13

Gggggaggcgggtctgggggaggcggaagtgggggaggaggaagc

### G4Sx3 Linker Amino Acid, SEQ ID NO: 10

GGGGSGGGGSGGGGS

CD3 scFv Nucleotide Sequence, SEQ ID NO: 14

Gaagttcagctgctcgaatccggcggcggccttgttcagccaggt
ggtagcttgaggctcagttgtgctgcatctgggtttacattctca
acttatgcgatgaactgggtgaggcaagcacctggaaagggactt
gagtgggtctcaagaattcgctccaaatacaacaactatgcgacg
tattacgcagactcagtgaaaggacggtttacgatatcacgggac
gattcaaagaatacactgtatttgcagatgaattctcttagggcc
gaagacactgccgtatactattgtgtacgccacggtaattttggc
aatagctatgtatcttggttcgcgtactggggccaaggcaccctt
gttactgtgtctagtgggggcggggggagtggtggcggaggaagt
ggcggggggggatctcaagcggtggttactcaagagccctccctt
actgtttctccggggggacggtcaccttgacttgtggcagttcaa
caggggcagtcactactagtaattatgcgaattgggtccaagaaa
agccgggccaagctttccggggactcatcggaggaacaaataaaa
gggcacccggcacacccgcgcgcttttccgggagtcttctgggcg
gcaaggcagccctcactctctctggggctcaacctgaggacgagg
ctgagtactattgtgccctctggtactcaaacctgtgggtctttg
gagggggaaccaagcttacggtcttg

CD3 scFv Amino Acid Sequence, SEQ ID NO: 15

EVQLLESGGGLVQPGGSLRLSCAASGFTFSTYAMNWVRQAPGKGL
EWVSRIRSKYNNYATYYADSVKGRFTISRDDSKNTLYLQMNSLRA
EDTAVYYCVRHGNFGNSYVSWFAYWGQGTLVTVSSGGGGSGGGGS
GGGGSQAVVTQEPSLTVSPGGTVTLTCGSSTGAVTTSNYANWVQE
KPGQAFRGLIGGTNKRAPGTPARFSGSLLGGKAALTLSGAQPEDE
AEYYCALWYSNLWVFGGGTKLTVL

### CD3 VH Nucleotide, SEQ ID NO: 16

Gaagttcagctgctcgaatccggcggcggccttgttcagccaggt
ggtagcttgaggctcagttgtgctgcatctgggtttacattctca
acttatgcgatgaactgggtgaggcaagcacctggaaagggactt
gagtgggtctcaagaattcgctccaaatacaacaactatgcgacg
tattacgcagactcagtgaaaggacggtttacgatatcacgggac
gattcaaagaatacactgtatttgcagatgaattctcttagggcc
gaagacactgccgtatactattgtgtacgccacggtaattttggc
aatagctatgtatcttggttcgcgtactggggccaaggcaccctt
gttactgtgtctagt

### CD3 VH Amino Acid, SEQ ID NO: 17

EVQLLESGGGLVQPGGSLRLSCAASGFTFSTYAMNWVRQAPGKGL
EWVSRIRSKYNNYATYYADSVKGRFTISRDDSKNTLYLQMNSLRA
EDTAVYYCVRHGNFGNSYVSWFAYWGQGTLVTVSS

### G4Sx3 Linker Nucleotide, SEQ ID NO: 18

ggg ggc ggg ggg agt ggt ggc gga gga agt ggc
ggg ggg gga tct

### G4Sx3 Linker Amino Acid, SEQ ID NO: 10

GGGGSGGGGSGGGGS

### CD3 VL Nucleotide, SEQ ID NO: 19

caagcggtggttactcaagagccctcccttactgtttctccgggg
ggacggtcaccttgacttgtggcagttcaacaggggcagtcacta
ctagtaattatgcgaattgggtccaagaaaagccgggccaagctt
tccggggactcatcggaggaacaaataaaagggcacccggcacac
ccgcgcgcttttccgggagtcttctgggcggcaaggcagccctca
ctctctctggggctcaacctgaggacgaggctgagtactattgtg
ccctctggtactcaaacctgtgggtctttggagggggaaccaagc
ttacggtcttg

### CD3 VL Amino Acid, SEQ ID NO: 20

QAVVTQEPSLTVSPGGTVTLTCGSSTGAVTTSNYANWVQEKPGQA
FRGLIGGTNKRAPGTPARFSGSLLGGKAALTLSGAQPEDEAEYYC
ALWYSNLWVFGGGTKLTVL

Human Fc with L234AL235AG329G, mutations are shown in bigger font and underlined

### Nucleotide, SEQ ID NO: 21

tctagagaaaacctgtattttcagggcacccacacgtgcccccct
tgcccagcacccgaa [symbol] ggtggcccatcagtgtttcttttt
cctccaaaaccaaaagacacactcatgatctcccggacgcctgag
gtgacctgtgtagtcgtagacgtatcccatgaggaccctgaagta
aagtttaactggtatgtagacggtgtggaagtacacaatgccaag
actaaaccaagagaggaacagtataacagcacctatagggtagtt
tccgtgctcaccgttctccaccaagattggcttaacggtaaagaa
tataaatgtaaggtgtcaaataaggcactc [symbol] gccccgatcgaa
aagaccatctctaaagcaaaaggacagcccagggagccacaagtc
tacaccctgcccccatcccgggatgagctgaccaagaaccaggtc
agcctgacctgcctggtcaaaggcttctatcccagcgacatcgcc
gtggagtgggagagcaatgggcagccggagaacaactacaagacc
acgcctcccgtgctggactccgacggctccttcttcctctacagc
aagctcaccgtggacaagagcaggtggcagcaggggaacgtcttc
tcatgctccgtgatgcatgaggctctgcacaaccactacacgcag
aagagcctctccctgtctcccgggaaatga

### Human Fc Amino Acid, SEQ ID NO: 22

SRENLYFQGTHTCPPCPAPE [symbol] GGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKAL [symbol] APIEKTISKAKGQPREPQV
YTLPPSRDELTKNQVSLTCLVKGFYPSDIAVEWESNGQPENNYKT
TPPVLDSDGSFFLYSKLTVDKSRWQQGNVFSCSVMHEALHNHYTQ
KSLSLSPGK

### Example 3. RNA Transcription of her-2-CD3 Mutant Human Fc Antibody

The sequence PMC2087 (template to make FIG. 1 structure) was inserted into a DNA vector, which is a template for in vitro RNA transcription.

Before in vitro transcription reaction, the sequence was linearized in the vector sequence at the end of poly A tail. During in vitro transcription reaction, the 5′Cap was added for increased stability of RNA. The RNA was checked on agarose gel and used to transfect 293 cells to generate Her-2-CD3-hFc antibody.

### Example 4. Her-2-CD3-hFc RNA-LNP Transfection Caused Binding with her-2-Positive SKOV-3 and T Cells

Her-2-CD3 human Fc mRNA was generated by in vitro transcription using DNA template shown in Example 2. Her-2-CD3 hFc RNA was embedded to LNP, the RNA-LNP-LNP was transfected to 293 S cells and supernatant was collected 72 hours after transfection.

FACS was performed 72 hours after transfection with supernatant collected from Her-2-CD3 RNA-LNP-transfected 293 cells using SKOV-3, CHO and T cells. FACS shows high binding of secreted antibody with SKOV-3 cells and T cells.

### Example 5. Transfection of her-2-CD3 RNA-LNP to Cancer Cells Resulted in Secretion of Functional Bispecific Antibody that Bound her-2-Positive Cells and CD3-Positive T Cells

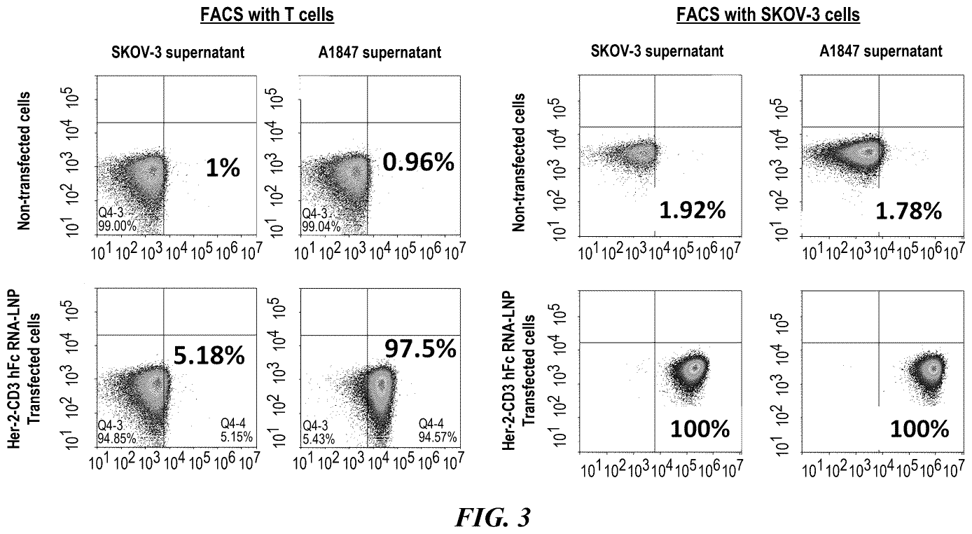

Her-2-CD3 hFc RNA-LNP was transfected to ovarian A1847 and SKOV-3 cancer cells. Supernatant collected from transfected cells showed binding with Her-2 positive SKOV-3 cells and T cells (FIG. 3). Binding with T cells was higher with supernatant collected from A1847 cells than from SKOV-3 cells. The results show that transfection of Her-2-CD3 RNA-LNP to cancer cells resulted in secretion of functional bispecific antibody that bound Her-2-positive cells and CD3-positive T cells.

### Example 6. Her-2-CD3 Human Fc mRNA-LNP-Transfected A1847 Cancer Cells Generated Bispecific Antibody Killing her-2 Positive Target Cells in the Presence of T Cells

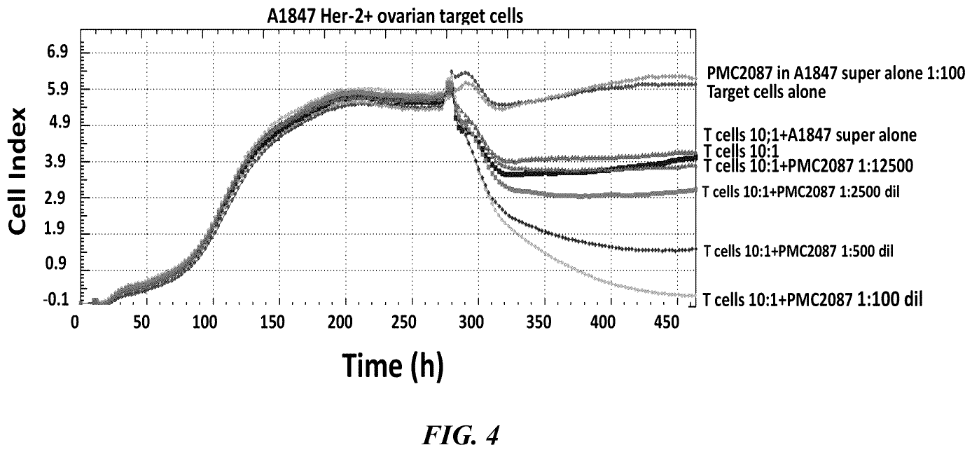

We transfected A1847 cells with Her-2-CD3 hFc RNA-LNP and collected supernatant with bispecific antibody to test killing of Her-2-positive ovarian cancer A1847 target cells. The supernatant with T cells killed target cells in a dose-dependent manner while supernatant with antibody alone or T cell alone did not kill target cells (FIG. 4). Her-2-negative CHO cells were not killed with the same dilutions of supernatant and T cells (not shown).

### Example 7. Her-2-CD3 Human Fc mRNA-LNP-Transfected A1847 Cancer Cells Generated Bispecific Antibody that Caused Secretion of IFN-Gamma Against her-2 Positive Target Cells

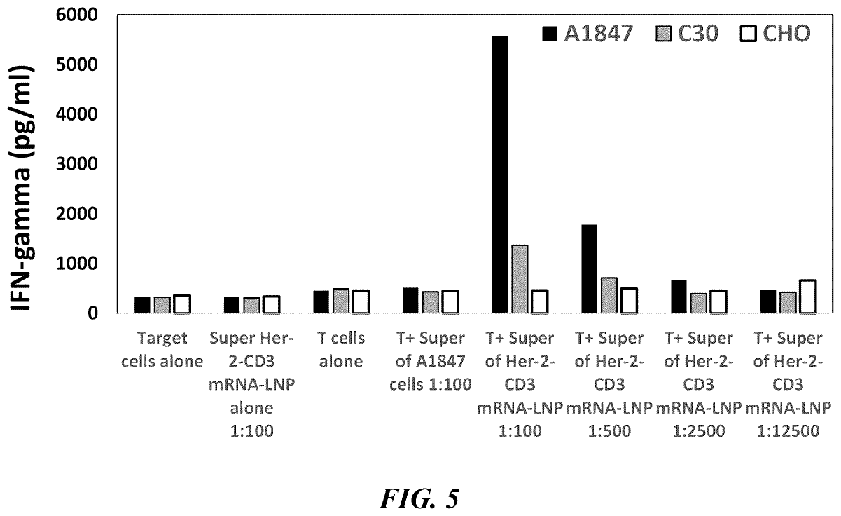

Her-2-CD3 hFc mRNA-LNP transfected cancer cells generated antibody that with T cells caused secretion of IFN-gamma against Her-2 positive cells but not against Her-2-negative CHO cells (FIG. 5).

### Example 8. Her-2 mRNA-LNP Intratumoral Injection Blocked Ovarian Tumor Growth In Vivo

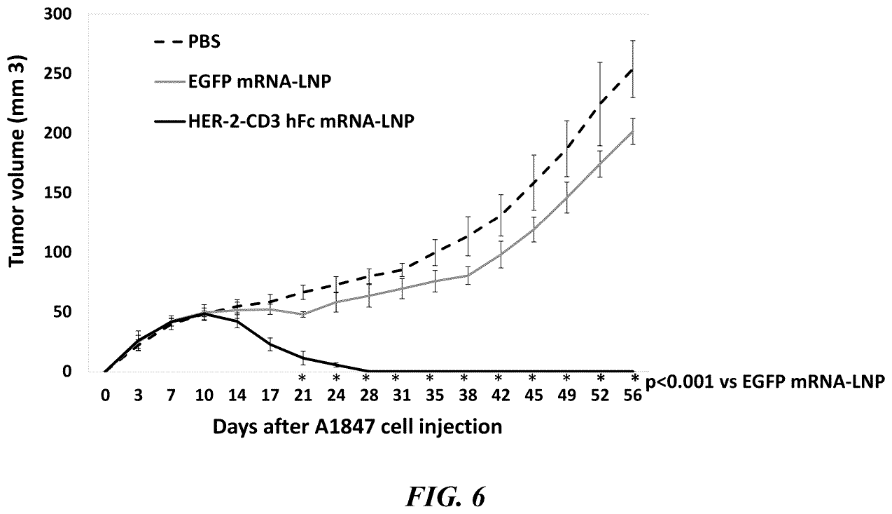

A1847 ovarian tumor cells (2×10⁶ cells/mice) were injected into NSG mice, and then Her-2-CD3 hFc mRNA mRNA-LNP or negative control EGFP mRNA-LNP (1 microg/mice) were injected 3 times at day 7, 14, 21 intratumorally. T cells (1×10⁷ cells/mice) were injected on day 9 intravenously. Her-2-CD3 hFc mRNA-LNP significantly decreased A1847 ovarian tumor growth (FIG. 6). The results show that Her-2-CD3 hFc mRNA can be delivered inside tumors to generate bispecific antibody with high efficacy against ovarian xenograft tumors.

What is claimed is:

1. A bispecific antigen-binding molecule comprising HER-2 VL having SEQ ID NO: 8, a first linker, HER-2 VH having SEQ ID NO: 12, a second linker, CD3 VH having SEQ ID NO: 17, a third linker, CD3 VL having SEQ ID NO: 20, and a human Fc domain, wherein the CH2 of the human Fc domain optionally comprises one or more amino acid substitutions selected from the group consisting of L234, L235, and P329 (EU numbering).

2. The bispecific antigen-binding molecule of claim 1, wherein the first, the second, and the third linkers are the same or different.

3. The bispecific antigen-binding molecule of claim 1, wherein each linker has the amino acid sequence of SEQ ID NO: 10.

4. The bispecific antigen-binding molecule of claim 1, wherein the human Fc domain has the amino acid sequence of SEQ ID NO: 22.

5. An isolated DNA sequence comprising (a) a promoter coding sequence, (b) 5′-UTR (untranslated region) coding sequence, (c) a coding sequence to encode the bispecific antigen-binding molecule of claim 1, (d) a 3′-UTR coding sequence, and (e) a poly A tail sequence.

6. The isolated DNA sequence of claim 5, having the nucleotide sequence of SEQ ID NO: 1.

7. Lipid nanoparticles (LNPs) have mRNA encapsulated, wherein the mRNA is transcribed from the isolated DNA sequence of claim 5.

8. A method for producing bispecific antigen-binding molecules in cells, comprising the steps of:
electroporating or transfecting the mRNA-encapsulated LNPs of claim 7 into cells, and
translating the mRNA in the cell to produce the bispecific antigen-binding molecule.

9. A method for treating cancer, comprising the steps of:
injecting the mRNA-encapsulated LNP of claim 7 to a tumor lesion of a subject having cancer.

10. The method of claim 9, which activates T cells and NK cells in the tumor microenvironment.

11. The method of claim 9, wherein said cancer is ovarian, pancreatic, or colon cancer.
